(19) United States
(12) Patent Application Publication
(10) Pub. No.: US 2021/0161695 A1
(43) Pub. Date: Jun. 3, 2021

(54) Plectrum or Pick Assistive Devices and Methods

(71) Applicant: Jett Manley, Sulphur, LA (US)

(72) Inventor: Jett Manley, Sulphur, LA (US)

(21) Appl. No.: 16/780,719

(22) Filed: Feb. 3, 2020

Related U.S. Application Data
(60) Provisional application No. 62/801,054, filed on Feb. 4, 2019.

(51) Int. Cl. A61F 4/00; G10D 3/173

(52) U.S. Cl. CPC A61F 4/00; G10D 3/173

(57) ABSTRACT

Assistive devices for picking a guitar or the like, in which preferably two fused rings each permit insertion of a finger or finger portion, and/or the fused rings are attached to a substantially planar pick support away from a centerline thereof, and/or and a flexible pick tip portion is integral with the pick support portion.

### CROSS-REFERENCE

Priority is claimed from U.S. application 62/801,054 filed Feb. 4, 2019, which is hereby incorporated by reference.

### BACKGROUND

The present application relates to a plectrum (pick) accessory, for playing guitars and other instruments with plucked strings, which is an assistive device for individuals with limited control over finger activation.

Note that the points discussed below may reflect the hindsight gained from the disclosed inventions, and are not necessarily admitted to be prior art.

Usually guitars are played with a pick. (A pick is properly known as a “plectrum,” and the two terms are regarded as synonyms in the present application). Some players use a “finger-style” to play without a pick, but a pick is indispensable in many styles of playing.

A pick usually has a shape somewhat similar to a fat triangle, or to an oval elongated to a point. Usually the pick includes some flex, to achieve a clean release from the plucked string. The pick is typically held between thumb and forefinger of the dominant hand, and picking or strumming is performed using some combination of rotation around the radius bone with flexion of the wrist or elbow joints. However, stable positioning of the pick requires a secure grip between thumb and forefinger, and not all players are able to do this.

Guitar is one of the most popular instruments in the world. 2.6 million acoustic and electric guitars were sold in the U.S. in 2017, which is 0.7% of the entire population; if these guitars have an average 10 years of use, that would imply that more than 1% of the entire population (and possibly more than 10%) play guitar.

Thus this is an extremely widespread activity. However, this activity, like others, can be affected by innate or acquired disabilities. Since musicmaking is very important to many peoples' lives, there is a need for assistive devices which will allow a player to bypass the strength requirements of the usual pick manipulation.

Some attempts at assistive devices have attached a pick to the (remnant) arm of a person who lacks a hand. However, this means that the picking motion is mostly provided by gross motion of the elbow joint, and this motion is not very subject to very fine motor control. Thus assistive devices like this provide some limited accommodation for this severe disability.

Other attempts at assistive devices have attached a pick to the (remnant) palmar structure of a hand. This provides some accommodation for missing or disabled fingers, but is still somewhat limited in the security of attachment and fine motor control.

Previously proposed picks are shown e.g. in U.S. Pat. Nos. 2,776,592, 3,442,169, 3,648,558, and 5,973,243, all of which are hereby incorporated by reference. Other examples of picks are shown in published US applications US20050045020, US20080148920, US20090056521, and US20090139384, all of which are also hereby incorporated by reference. In addition, all US patents and published US applications which reference any of the foregoing 8 patents and applications are also hereby incorporated by reference.

### Plectrum or Pick Assistive Devices and Methods

The present application teaches, among other innovations, an assistive device for picking a guitar or the like, in which two fused rings each permit insertion of a finger or finger portion, and only one of the fused rings is attached to a substantially planar pick support away from a centerline thereof, and a flexible pick tip portion is integral with the pick support portion and lies generally perpendicular to the plane defined by the rings, and extends out beyond the user's thumb (if present).

The present application teaches, among other innovations, an assistive device for picking a guitar or the like, comprising a single body of material in which fused rings permit insertion of fingers or stubs, and extend integrally to provide a flexible pick tip portion.

The present application teaches, among other innovations, an assistive device for picking a guitar or the like, comprising a single body of material in which fused rings permit insertion of fingers or stubs, and extend integrally to provide a pick support portion, and wherein the pick support portion can hold a flexible pick.

The present application teaches, among other innovations, an assistive device for picking a guitar or the like, in which fused rings permit insertion of fingers or stubs, and the fused rings are attached to a pick support away from a centerline thereof, and a flexible pick tip portion is integral with the pick support portion.

The present application teaches, among other innovations, an assistive device for picking a guitar or the like, comprising a single body of material in which fused rings permit insertion of fingers or stubs, and extend integrally to provide a flexible pick tip portion which lies generally perpendicular to the plane defined by the rings.

The present application teaches, among other innovations, an assistive device for picking a guitar or the like, in which two or more fused rings permit insertion of fingers or stubs, and the fused rings are attached to a pick support away from a centerline thereof, and a flexible pick tip portion is integral with the pick support portion and extends out beyond the user's thumb (if present).

The present application teaches, among other innovations, an assistive device for picking a guitar or the like, in which two or more fused rings each permit insertion of fingers or stubs, and only one of the fused rings is attached to a substantially planar pick support portion away from a centerline thereof, and a flexible pick tip portion is integral with the pick support portion and extends out beyond the user's thumb (if present).

The present application teaches, among other innovations, an assistive device for picking a guitar or the like, in which two or more fused rings each permit insertion of fingers or stubs, and only one of the fused rings is attached to a substantially planar pick support portion away from a centerline thereof, and a flexible pick tip portion is integral with the pick support portion and extends out beyond the user's thumb (if present).

The present application teaches, among other innovations, an assistive device for picking a guitar or the like, in which two or more fused rings each permit insertion of fingers or stubs, and only one of the fused rings is attached to a substantially planar pick support portion away from a centerline thereof, and a flexible pick tip portion is integral with the pick support portion and extends out beyond the user's thumb (if present).

The present application teaches, among other innovations, an assistive device for picking a guitar or the like, in which two or more fused rings each permit insertion of fingers or stubs, and only one of the fused rings is attached to a substantially planar pick support portion away from a centerline thereof, and a flexible pick tip portion is integral with the pick support portion and extends out beyond the user's thumb (if present).

The present application teaches, among other innovations, an assistive device for picking a guitar or the like, in which two or more fused rings each permit insertion of fingers or stubs, and only one of the fused rings is attached to a substantially planar pick support portion away from a centerline thereof, and a flexible pick tip portion is integral with the pick support portion and extends out beyond the user's thumb (if present).

Other assistive picking devices are also disclosed, in which a formed piece, which includes a thinned extension providing a flexible pick, is attached to available anatomy using conventional straps or prosthesis fittings. Here too the use of a single piece which includes both a substantially rigid body and also a flexible pick extension advantageously provides a hybrid support+pick structure.

The present application also discloses methods for using and methods for making each of the above devices.

### BRIEF DESCRIPTION OF THE DRAWINGS

The disclosed inventions will be described with reference to the accompanying drawings, which show important sample embodiments and which are incorporated in the specification hereof by reference, wherein:

FIG. 1 schematically shows a first example of an assistive device for picking a guitar or other manually plucked musical instrument.

FIG. 2 and FIG. 3 show side views of the device of FIG. 1

FIGS. 4-7 schematically show a second example of an assistive device for picking a guitar or other manually plucked musical instrument.

FIG. 8 schematically shows a third example of an assistive device for picking a guitar or other manually plucked musical instrument. Note that the drawing shows a user with a functional thumb but no other digits.

FIG. 9 schematically shows a fourth example of an assistive device for picking a guitar or other manually plucked musical instrument. Note that the drawing shows a user with no functional digits.

FIG. 10 schematically shows a fifth example of an assistive device for picking a guitar or other manually plucked musical instrument. Note that the drawing shows a user with no functional wrist joint nor hand, e.g. a brachial amputee.

FIG. 11 schematically shows a sixth example of an assistive device for picking a guitar or other manually plucked musical instrument. Note that the pick portion, in this example, is coplanar with the rings.

### DETAILED DESCRIPTION OF SAMPLE EMBODIMENTS

The numerous innovative teachings of the present application will be described with particular reference to presently preferred embodiments (by way of example, and not of limitation). The present application describes several inventions, and none of the statements below should be taken as limiting the claims generally.

The present application discloses several new versions of an assistive device for picking a guitar or other manually plucked musical instrument. These are most suitable for situations where a normal plectrum (pick) does not suit the individual's needs when playing a certain instrument. However, note also that some of these can also be advantageous with normal users, especially to accommodate high humidity, or muscle weakness, or recovery from injury.

In this case it is 3D printed, but it could be made out of any pliable material/s. This assistive device consists of one or more offset rings that wrap around whatever part of the body that can enable the user to play more effectively or to have the capability to play at all. For example, if a person has radioulnar synostosis or is lacking distal extremities, specifically fingers, hands, toes, or feet, the circumference and diameter of the existing body part is measured to find the best oval or circle ring fit for the user. The offset ring or rings attach to a reinforcement base that holds the plectrum (pick) in place so the user can play their instrument without the restrictions of holding a normal plectrum (pick).

### FIRST EXAMPLE

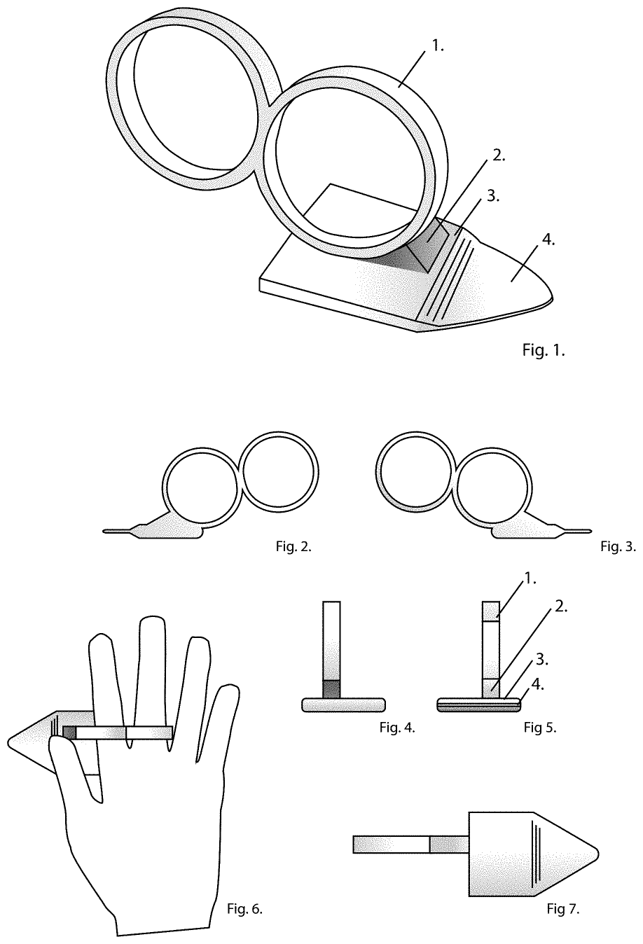

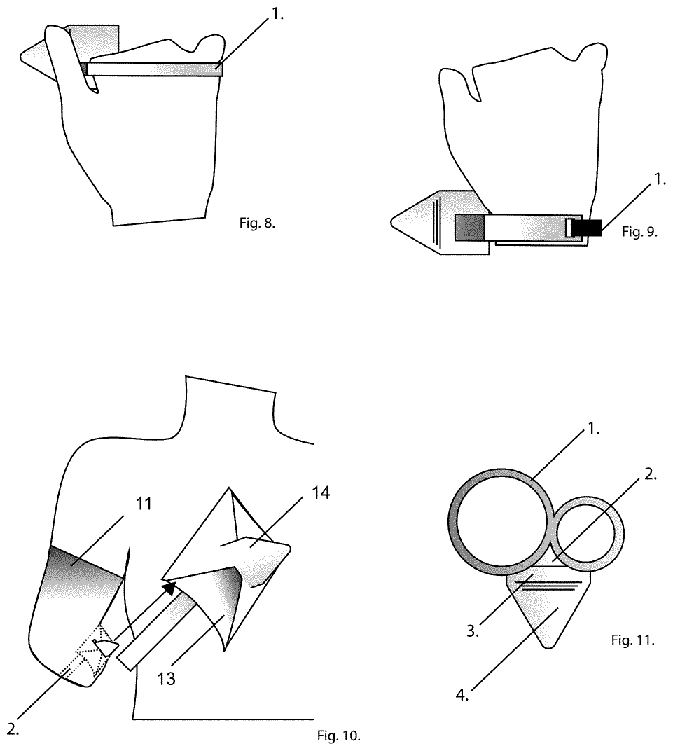

FIG. 1 is a 3-D elevational view of a guitar pick holder showing a first implementation. Fused rings 1 are sized to accommodate a user's first and second fingers. These can optionally be sized differently for different individual user's needs. These are preferably made of a polymer material, as described below.

An attachment portion 2 mechanically connects the rings 1 to a pick support 3. Preferably the rings 1, attachment portion 2, and pick support 3 are all made of the same polymer material, and are all formed simultaneously, as an integral body, in a single 3D printing operation.

In this example the attachment portion 2 is located asymmetrically, i.e. not along the centerline of the pick support 3, but the rings 1 are not. Note that the attachment portion 2 serves partly to provide support and positioning for a user's thumb.

Alternatively, as described below, both the attachment portion and the rings can be offset from the centerline of the pick support 3.

The pick support 3, in this example, extends to form a flexible pick tip 4. The pick tip 4 is thinner than and pick support.3, and hence more flexible. Thus the pick tip 4 can be dimensioned to match any desired degree of flexibility. (Picks are normally sold in a range of weights, so that they can be matched to the string being plucked and also to the user's preference.)

FIG. 2 and FIG. 3 show right and left elevations of the device of FIG. 1. Note that, when the rings 1 and/or the attachment portion 2 are offset from the centerline of the pick support 3, the assistive device can be made in two different versions for right-handed and left-handed users.

### SECOND EXAMPLE

FIG. 4 shows an alternative version of the device of FIG. 1. Note that the rings 1 and the attachment portion 2 are both offset from the centerline of the pick support 3. FIG. 4 is a rear view, in which the flexible pick portion 4 is not visible.

FIG. 5 is a front view of the assistive device of FIG. 4, showing the openings of the rings in phantom. Note that the flexible pick portion 4 is visible in this drawing.

FIG. 6 is a top view of the device of FIG. 4, showing the device in relation to a user's hand.

FIG. 7 is a bottom view of the device of FIG. 4.

### THIRD EXAMPLE

FIG. 8 schematically shows a third example of an assistive device for picking a guitar or other manually plucked musical instrument. Note that the drawing shows a user with a functional thumb but no other digits.

### FOURTH EXAMPLE

FIG. 9 schematically shows a fourth example of an assistive device for picking a guitar or other manually plucked musical instrument. Note that the drawing shows a user with no functional digits.

### FIFTH EXAMPLE

FIG. 10 schematically shows a fifth example of an assistive device for picking a guitar or other manually plucked musical instrument. Note that the drawing shows a user with no functional wrist joint nor hand, e.g. a brachial amputee and/or congenital missing lower arm.

In this example, the holding sleeve 11 is made of tight-stretching material, and includes a non-slip holding slit (similar to a buttonhole) to permit a support 13 to extend a pick 14 (or flexible pick portion 14). A Velcro or elastic attachment accommodates the available arm portion. Preferably the support 13 all fits inside of holding sleeve 14. The user can twist the sleeve 11, and thereby relocate the support 13, when and ring for effective directional placement.

### SIXTH EXAMPLE

FIG. 11 schematically shows a sixth example of an assistive device for picking a guitar or other manually plucked musical instrument. Note that the pick portion, in this example, is coplanar with the rings. This is particularly advantageous for toe playing.

### Manufacture

Preferably the examples of FIGS. 1-7 and 11 are entirely formed by 3D printing.

Similarly, the pick support and flexible pick tip of FIGS. 8-10 are preferably formed entirely by 3D printing, so that the pick support and flexible pick tip are integral parts of a single mass.

### Advantages

The disclosed innovations, in various embodiments, provide one or more of at least the following advantages. However, not all of these advantages result from every one of the innovations disclosed, and this list of advantages does not limit the various claimed inventions.
Guitar playing is possible without fully functional fingers.
The first and second examples above provide a mild or transitional assistive device, which can compensate for some degree of user disability while providing functionality which is actually equal to or very slightly less than normal pick handling.

According to some but not necessarily all embodiments, there is provided: An assistive device for picking a stringed musical instrument, comprising: two fused rings which each permit insertion of a finger or finger portion, a pick support which is mechanically connected to the fused rings, and a flexible pick tip portion which is integral with the pick support.

According to some but not necessarily all embodiments, there is provided: An assistive device for picking a stringed musical instrument, comprising: rings which each permit insertion of a finger portion, and a flexible pick tip portion; wherein the rings and the pick tip portion are parts of a single integral body of material.

According to some but not necessarily all embodiments, there is provided: An assistive device for picking a stringed musical instrument, comprising a single body of material in which fused rings which permit insertion of fingers or stubs, and are integrally connected to a pick support portion which can hold a flexible pick.

According to some but not necessarily all embodiments, there is provided: An assistive device for picking a stringed musical instrument, comprising: rings which each permit insertion of a finger portion, and a flexible pick tip portion; wherein the rings and the pick tip portion are parts of a single integral body of material; and wherein the flexible pick tip portion lies generally perpendicular to a plane defined by the rings.

According to some but not necessarily all embodiments, there is provided: Assistive devices for picking a guitar or the like, in which preferably two fused rings each permit insertion of a finger or finger portion, and/or the fused rings are attached to a substantially planar pick support away from a centerline thereof, and/or and a flexible pick tip portion is integral with the pick support portion.

### Modifications and Variations

As will be recognized by those skilled in the art, the innovative concepts described in the present application can be modified and varied over a tremendous range of applications, and accordingly the scope of patented subject matter is not limited by any of the specific exemplary teachings given. It is intended to embrace all such alternatives, modifications and variations that fall within the spirit and broad scope of the appended claims.

While the above description primarily refers to use with a guitar, the disclosed plectrums can also be used with other plucked instruments, including e.g. but not limited to: bass guitar, balalaika, ukulele, mandolin, lute, zither, banjolele, dobro, etc.

Note that, especially in embodiments like FIGS. 8-10, the length of the pick support portion can be varied to vary the positioning of the pick. Where the length of the extant anatomy is less than that of an unimpaired arm, this can be useful to get the pick positioned optimally.

Note that the fused rings do not have to form a perfect Figure-8. For example, while the sample embodiments herein show mutually separate fingerholes, the fingerholes do not have to be totally separated by a divider as shown. Also, the thickness of the divider between fingerholes does not have to be the same as the thickness of the outer walls of the fingerholes.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

The claims as filed are intended to be as comprehensive as possible, and NO subject matter is intentionally relinquished, dedicated, or abandoned.

1. An assistive device for picking a stringed musical instrument, comprising:
two fused rings which each permit insertion of a finger or finger portion,
a pick support which is mechanically connected to the fused rings, and
a flexible pick tip portion which is integral with the pick support.

2. The device of claim 1, wherein the fused rings and the pick support are integral parts of a single body of material.

3. The device of claim 1, wherein the fused rings are joined to the pick support through an attachment portion, and wherein the fused rings, the attachment portion, and the pick support, are all integral parts of a single body of material.

4. The device of claim 1, wherein the fused rings have respective internal spaces which are all mutually separate.

5. The device of claim 1, wherein the fused rings are offset from a centerline of the pick support.

6. An assistive device for picking a stringed musical instrument, comprising:
rings which each permit insertion of a finger portion, and
a flexible pick tip portion;
wherein the rings and the pick tip portion are parts of a single integral body of material.

7. The device of claim 6, wherein the rings have respective internal spaces which are all mutually separate.

8. The device of claim 6, wherein the rings are all fused together.

9. The device of claim 6, wherein the fused rings are joined to a pick support through an attachment portion, and wherein the fused rings, the attachment portion, and the pick support, are all integral parts of a single body of material.

10. The device of claim 7, wherein the fused rings are joined to a pick support, and are offset from a centerline of the pick support.

11. An assistive device for picking a stringed musical instrument, comprising a single body of material in which
fused rings which permit insertion of fingers or stubs, and are integrally connected to
a pick support portion which can hold a flexible pick.

12. The device of claim 11, wherein the rings have respective internal spaces which are all mutually separate.

13. The device of claim 11, wherein the pick support portion extends to form a flexible pick tip which is integral with and thinner than the pick support portion.

14. The device of claim 11, wherein the rings have respective geometries which are all fused together.

15. The device of claim 11, wherein the fused rings are joined to a pick support through an attachment portion, and wherein the fused rings, the attachment portion, and the pick support, are all integral parts of a single body of material.

16. The device of claim 11, wherein the fused rings are joined to a pick support, and are offset from a centerline of the pick support.

17-22. (canceled)
